(19) United States
(12) Patent Application Publication
(10) Pub. No.: US 2026/0148418 A1
(43) Pub. Date: May 28, 2026

(54) METHOD FOR DETERMINING A POSE OF A STEREO BASE OF A STEREO SYSTEM OF A MEDICAL VISUALIZATION SYSTEM, AND MEDICAL VISUALIZATION SYSTEM

(71) Applicant: Carl Zeiss Meditec AG, Jena (DE)

(72) Inventors: Michael Wirth, Aalen (DE); Richard Bäumer, Urbach (DE); Manuela Warnecke, Aalen (DE); Christian Beder, Aalen (DE)

(21) Appl. No.: 19/397,823

(22) Filed: Nov. 21, 2025

(30) Foreign Application Priority Data
Nov. 22, 2024 (DE) 10 2024 134 514.4

(51) Int. Cl. G06T 7/80; A61B 90/20; G06T 5/50; G06T 5/80

(52) U.S. Cl. CPC G06T 7/85; G06T 5/50; G06T 5/80; A61B 90/20; G06T 2207/10012; G06T 2207/10056; G06T 2207/30004; G06T 2207/30244

(57) ABSTRACT

A medical visualization system and a method for determining a pose of a stereo base of a stereo system of the medical visualization system are provided. The method includes setting a first relative pose between a calibration object and a focal pose of the medical visualization system, generating a reference image with a first image capturing device, determining a reference image-specific zoom center in the reference image, generating a remaining image with a second capturing device, and determining a pose of a back-projected zoom center as the pose of the reference image-specific zoom center back-projected into the remaining image. The pose of the stereo base is determined depending on poses of the back-projected zoom center in the remaining image, or the pose of the stereo base is determined depending on poses of the image-specific zoom center and the back-projected zoom center in the remaining image.

### CROSS REFERENCE TO RELATED APPLICATIONS

This application claims priority to German patent application DE 10 2024 134 514.4, filed Nov. 22, 2024, the entire content of which is incorporated herein by reference.

### TECHNICAL FIELD

The disclosure relates to a method for determining a pose of a stereo base of a stereo system of a medical visualization system, and to a medical visualization system.

### BACKGROUND

Surgical microscopes, inter alia, are used to prepare and perform medical operations on a patient. During a treatment, such surgical microscopes are used by a user, e.g., a surgeon or an assistant, to provide a representation, in particular a magnified representation, of an examination region, in or on the situs of the patient. To this end, a surgical microscope may include an objective or an objective system for generating a real optical image representation of the examination region. The objective may include optical elements, e.g., a lens, for beam guidance and/or beam shaping and/or beam steering.

Surgical microscopes find use in medical facilities, but also in laboratories or in industrial applications. Exemplary medical fields of application are neurosurgery, eye surgery, Otolaryngology (ENT) surgery, plastic or reconstructive surgery, and orthopedic surgery. This list is not exhaustive. In general, they are used in all areas of surgery which require a magnified and high-resolution view of the operating field in order to perform precise interventions.

A distinction can be made between analog and digital surgical microscopes. In contrast to digital surgical microscopes, analog surgical microscopes do not capture images which are subsequently displayed, e.g., on a screen for the magnified representation of the examination region, but instead offer a directly visually capturable magnification of the examination region for the user. In this context, radiation reflected or scattered off the use region passes through the objective into at least one beam path and to at least one output portion, through or into which the user gazes in order to visually capture the radiation and hence also the generally magnified representation of the examination region. One exemplary embodiment of an output portion is a so-called eyepiece, into or through which the user gazes in order to optically capture the examination region with at least one eye. Such an eyepiece is usually arranged on a microscope head. Analog surgical microscopes generally also include handles for positioning arranged on a microscope head, such handles also having operator control elements for controlling the surgical microscope, e.g., in the form of switches.

Digital surgical microscopes include exactly or at least one image capturing device for microscopic imaging, which captures radiation in a beam path of the surgical microscope in order to generate an image, in particular a magnified image, wherein this image can be displayed to the user or else to multiple users on one or more display device(s). Consequently, a high-resolution visualization can be made possible. The image can be generated in the form of an image signal, in particular a transmittable image signal, which encodes or represents the image. In contrast to analog surgical microscopes, purely digital surgical microscopes do not include any output portion for visually capturable radiation, i.e., do not include an eyepiece in particular. In that case, an image signal can be transmitted in the form of a data signal, in particular in wired or wireless fashion.

Digital surgical microscopes allow the recording of images and videos, and also the storing and further processing thereof. By applying image processing methods, it is possible to adapt contrast, brightness and other parameters in order to optimize an image quality of the images generated. Hybrid surgical microscopes can include both at least one image capturing device and at least one output portion. For example, the radiation guided in a beam path of the surgical microscope can be split using a beam splitter, with a first component being guided to the output portion and a further component being captured by the at least one image capturing device.

Also known are stereo surgical microscopes, which generally include two separate beam paths for beam guidance and provide the user with a depth impression of the examination region. For this purpose, the beams guided in the two beam paths can be visually captured by the user via output portions. Digital surgical microscopes have, alternatively or additionally, two image capturing devices, each of which records the beams in one of the beam paths in order to generate an image, wherein the user is then provided with a three-dimensional image made from the two images, which may also be referred to as corresponding images, via a suitable display device. The image recording devices are part of a stereo (camera) system.

In order to ensure a correct representation, an exact adjustment of the stereo camera system is required, wherein the adjustment ensures a correct representation of a three-dimensional image resulting from a superimposition of the images from the two beam paths. In order to achieve such a correct three-dimensional image, it may be necessary for the two images to image the same object point in the image centers, for example.

Another important parameter for providing a correct three-dimensional image is the orientation of the image capturing devices with respect to one another. For example, it may be desirable for the sensor surfaces of the two image capturing devices to be arranged parallel to one another and in particular in the same plane. It may thus be desirable for surface normals of the sensor surfaces to be oriented parallel to one another.

In order to avoid a reduction in the representation quality owing to distortions, in particular perspective distortions, it is additionally desirable to arrange the sensor surfaces of the image capturing devices in such a way that a first axis of symmetry of a sensor surface, which may also be referred to as a vertical axis of symmetry, is oriented perpendicular to a stereo base and a further axis of symmetry of the sensor surface, which may also be referred to as a horizontal axis of symmetry, is oriented parallel to the stereo base. For this purpose, it may be desirable to reliably and accurately determine the stereo base of the stereo system in order then to adapt the arrangement of the sensor surfaces or to take into account the orientations of the axes of symmetry relative to the stereo base in the image processing for determining the three-dimensional representation.

### SUMMARY

It is therefore an object of the disclosure to provide a method for determining a pose of a stereo base of a stereo system of a medical visualization system and a medical visualization system, in particular without an external aid, which allow an easily implementable and reliable determination of the stereo base, in order then to ensure a correct representation of a stereo image.

The object is achieved by a method for determining a pose of a stereo base of a stereo system of a medical visualization system, in particular a rotational pose of the stereo base, more particularly in an image coordinate system. The visualization system includes a stereo surgical microscope or can be formed by the stereo surgical microscope. Constituent parts of the medical visualization system explained hereinafter can be constituent parts of the stereo surgical microscope or constituent parts that are formed differently from the stereo surgical microscope. Surgical microscopes and their technical features have already been briefly explained in the introduction. The stereo surgical microscope can be a digital surgical microscope, in particular a hybrid or a purely digital surgical microscope. Such a visualization system, in particular the surgical microscope, can include a microscope head. The objective explained in the introduction can be integrated into the microscope head or secured, in particular releasably secured, to the latter. In this case, the objective can be arranged in a fixed position relative to the microscope head. In addition to the objective, the microscope head can likewise include or form at least one beam path for the microscopic imaging and/or further optical elements for beam guidance and/or beam shaping and/or beam deflection. The visualization system can have a fixed or an adjustable focal length.

In addition to the surgical microscope, the medical visualization system can include a stand for mounting the surgical microscope. The surgical microscope, in particular the microscope head, can be mechanically secured to the stand, in particular at a free end, and thus can form an end effector of the stand. The stand is configured such that it allows a movement of the surgical microscope in space, in particular with at least one degree of freedom, typically with six degrees of freedom, wherein a degree of freedom can be a translational or a rotational degree of freedom. Moreover, the stand can include at least one drive device for moving the surgical microscope. Such a drive device can be a servo motor, for example. Of course, the stand can also include means for transmitting forces/moments, for example gear units. In particular, it is possible for the at least one drive device to be controlled such that the surgical microscope carries out a desired movement and thus a desired change in pose in space or adopts a desired pose, i.e., a position and/or orientation, in space. For example, the at least one drive device can be controlled such that an optical axis of an objective of the surgical microscope adopts a desired orientation. Furthermore, the at least one drive device can be controlled such that a reference point of the surgical microscope, for example a focal point, is positioned at a desired position in space. A target pose can be specified by a user or by some other superordinate system. Methods for controlling the at least one drive device depending on a target pose and a kinematic structure of the stand are known here to a person skilled in the art.

The medical visualization system includes a stereo system having a first image capturing device and a further image capturing device, which serve for microscopic, i.e., for magnifying, imaging. An image capturing device can include a complementary metal-oxide-semiconductor (CMOS) or charge-coupled device (CCD) sensor. The visualization system, in particular the microscope head, can include or form two beam paths, namely a first beam path for the beams captured by the first image capturing device and a further beam path for the beams captured by the further image capturing device.

The stereo base can denote a connecting line of the centers of the beam paths in a cross-sectional plane perpendicular to the central axis of a beam path. In this case, the central axes of the beam paths are oriented parallel at least in portions. The cross-sectional plane can be arranged at a capturing device end of a beam path, wherein the central axes of the beam paths can also be oriented parallel to one another at this end. The beam paths can be part of a so-called video channel. The stereo base is not variable when the beam paths have been installed.

Furthermore, the medical visualization system, in particular the stereo surgical microscope, can include one or more of the elements listed below:
at least one white-light illumination device,
at least one infrared illumination device,
at least one fluorescence illumination device for exciting fluorescence,
at least one beam filter for providing excitation radiation with wavelengths from a broader spectrum, e.g., the spectrum from the white-light illumination device,
at least one fluorescence detection device for detecting fluorescence,
at least one filter device for filtering radiation from a broader spectrum, e.g., for capture by an image capturing device for microscopic imaging,
at least one input device for operator control,
at least one interface for data transfer to or from a further system or a further device, and
at least one storage device for storing signals and/or information, in particular in retrievable fashion.

The method includes the following steps.

In a setting step, a first relative pose between a calibration object and a focal pose of the medical visualization system is set. This setting can be effected by setting the focal pose, in particular with a setting depending on a contrast evaluation or with other setting methods. It is possible for the first relative pose to be determined after its setting, in particular depending on a contrast evaluation or with other methods for determining the focal pose.

The calibration object can be an optically capturable calibration object. A calibration object can be a two- or three-dimensional object. For example, a three-dimensional calibration object can include two Charuco boards oriented at an angle, in particular perpendicularly, to one another. In particular, the calibration object can include at least one optically capturable, in other words imageable, feature. The latter, too, can be a two- or three-dimensional feature. For example, such a feature can be a pattern, e.g., in the form of a barcode. Typically, the calibration object includes a plurality of such features, which however are formed differently from one another. In this case, a feature can be imaged into an image point set including at least one, typically more than one, image point. The calibration object can be of planar design, wherein the features are arranged on or formed by a surface of the planar calibration object.

In a generating step, an image is generated with one of the image capturing devices of the stereo system, i.e., the first or the further image capturing device, wherein this image serves as a reference image.

In a first zoom center determining step, a zoom center in the reference image is determined, i.e., the reference image-specific zoom center.

The zoom center denotes an image point that always images the same object portion during a change in a zoom value, in particular a beam path-specific zoom value, i.e., during zooming, of the medical visualization system. In other words, the zoom center is an image point around which the image is magnified or reduced when the zoom value is changed. In yet further words, the zoom center can represent the central axis or axis of symmetry of the respective beam path projected into the respective image. The zoom center is specified by a magnification system for setting different zoom values (zoom levels) or is a property of this system. It can change if there is a change in an orientation of a magnification system for setting different zoom values (zoom levels) relative to the sensor surface of the image capturing device that generates the corresponding image.

The zoom center can be determined for both beam paths. As explained above, the central axes of the beam paths can be oriented parallel at least in portions.

For determining the zoom center, at least two images can be generated with the image capturing device which generates the reference image, wherein the at least two images are generated with different zoom values in each case. One of these images can be the reference image. Then, by evaluation of these at least two images, the zoom center can be determined, in particular as the image point which images the same object portion in each of the at least two images.

In a further generating step, an image is generated with a further image capturing device of the stereo system, i.e., the remaining image capturing device, wherein this image serves as a remaining image. The reference image and the remaining image can also be recorded simultaneously.

The images, i.e., the reference image and the remaining image, can be encoded or represented in the form of image signals.

In a further determining step, a (image) pose of the reference image-specific zoom center back-projected into the remaining image is determined. The back-projected zoom center denotes the (image) pose of the reference image-specific zoom center in the remaining image.

In other words, in the remaining image, the image point or the set of image points which image the point or portion of the calibration object which is imaged into the zoom center of the reference image is thus determined. This can also be referred to as a (back) projection of the zoom center into the remaining image. In yet further words, the image point or image region of the remaining image that corresponds to the zoom center can be determined.

The zoom center in the remaining image can be determined in an image-based manner, especially by evaluation of the remaining image. For this purpose, a method for feature detection and, if appropriate, feature identification can be used.

According to a first alternative according to the disclosure, in a change step the relative pose between the calibration object and a focal pose of the medical visualization system is changed at least once, wherein after the or each change or for each set relative pose, the sequence including the further generating step and the further determining step is repeated.

A pose determining step involves determining the pose, in particular the rotational pose, of the stereo base depending on the poses of the zoom center in the remaining image. In particular, the pose of the stereo base can be determined in an image coordinate system of the remaining image. It can thus be assumed that the pose of the zoom center in the remaining image ideally runs along a straight line when the relative pose changes. The pose of this straight line, in particular in the image coordinate system, represents information about the pose of the stereo base. In particular, the gradient of the straight line or an angle between a horizontal image axis or the horizontal axis of symmetry and the straight line can represent an angle by which the sensor surface is rotated in relation to its target pose relative to the stereo base. In other words, the pose of the stereo base can be determined as the angle by which the sensor surface must be rotated in order that the horizontal image axis is oriented parallel to the explained straight line representing the stereo base.

It should be expected that the pose of the zoom centers runs along a straight line oriented parallel to the horizontal image axis or axis of symmetry explained above if the sensor surfaces of the image capturing devices are arranged in a target pose with respect to the stereo base. In the target pose of the sensor surface, the latter is arranged in such a way that the vertical axis of symmetry (and thus also a vertical image axis) is oriented perpendicularly to the stereo base and the horizontal axis of symmetry (and thus also the horizontal image axis) is oriented parallel to the stereo base. The pose of the stereo base can be determined relative to one of the sensor surfaces, in particular in a reference coordinate system of the sensor surface, wherein the reference coordinate system of the sensor surface can be formed by the horizontal and vertical image axes.

If the pose of the zoom centers runs along a straight line that is not oriented parallel to the horizontal axis of symmetry or image axis explained above, an actual pose of the sensor surface, in particular the sensor surface of the remaining image, deviates from its target pose. In this case, depending on an orientation of the straight line relative to the horizontal axis of symmetry, in particular an angle, information about the deviation of the actual pose of the at least one sensor surface from its target pose can be determined. Depending on this information, e.g., the arrangement of the sensor surface, in particular its position and/or orientation, can then be changed, in particular in order to reduce the deviation of the actual pose from the target pose. However, the information can also be taken into account in image processing for determining the three-dimensional representation.

According to a second alternative according to the disclosure, in particular after the further determining step, a pose of an image-specific zoom center in the remaining image is determined in a further zoom center determining step. This can be done according to the explanations for determining the reference image-specific zoom center. A pose determining step then involves determining the pose of the stereo base depending on the pose of the image-specific zoom center and the pose of the back-projected reference image-specific zoom center in the remaining image.

It can thus be assumed that the pose of a straight line connecting the image-specific zoom center and the back-projected reference image-specific zoom center represents information about the pose of the stereo base, wherein the pose of the straight line is determined in particular in the image coordinate system of the remaining image. As with regard to the first alternative according to an aspect of the disclosure, in particular, the gradient of the straight line or an angle between a horizontal image axis or the horizontal axis of symmetry and the straight line can represent an angle by which the sensor surface is rotated in relation to its target pose relative to the stereo base. Therefore, the explanations applicable with regard to this straight line are the same as those applicable with regard to the straight line along which the zoom centers determined according to the first alternative according to the disclosure progress.

Typically, the entire method is carried out repeatedly, wherein the first image forms the reference image in a first method pass and the further image forms the reference image in the second method pass. The pose of the stereo base relative to the first sensor surface can then be determined in the first method pass, and the pose of the stereo base relative to the further sensor surface in the second method pass.

The proposed method advantageously enables an easily implementable and reliable determination of the stereo base, which can then be used for the correct representation of a stereo image.

In particular in order to minimize an effect of erroneous target poses when determining the pose of the stereo base, the method can be carried out for a plurality of different zoom levels, in particular for two or more than two different zoom levels, wherein a resulting pose is then determined depending on the zoom level-specific poses of the stereo base, e.g., by averaging. Such erroneous target poses may occur if beam paths are not oriented parallel or lenses of the explained magnification system are tilted. In such a determination of a resulting pose, however, the zoom level-specific poses which deviate by more than a predetermined amount from the average of the remaining poses cannot be taken into account. The zoom level-specific poses determined on the basis of zoom centers which deviate by more than a predetermined amount from the average of the remaining zoom centers cannot be taken into account either. Such a deviation can also be used for fault diagnosis for the magnification system.

The proposed method is suitable for stereo systems in which the optical axes or surface normals of sensor surfaces of the two image capturing devices of the stereo system are arranged parallel or nonparallel, such as, e.g., in a Greenough-type microscope.

In a further exemplary embodiment, at least two images are generated by the image capturing device which generates the reference image, wherein the at least two images are generated with different zoom values in each case, wherein the zoom center is determined by evaluation of these at least two images. This has been explained above and advantageously enables a particularly reliable and accurate determination of the zoom center. In this case, the generation of the abovementioned remaining images and the images for determining the zoom center can be carried out at least partly simultaneously. Moreover, images generated with different zoom values and/or different relative poses can be stored and then subsequently evaluated.

In a further exemplary embodiment, a pose of the zoom center in the reference image is determined, wherein determining the pose of the back-projected zoom center is carried out depending on this pose in the reference image. In other words, when determining the pose in the remaining image, information about the pose of the zoom center in the reference image can be taken into account, especially quantitatively. The pose of the zoom center can be determined as an image coordinate.

The pose of the zoom center can be determined with a feature detection method.

It is possible, for example, to determine the pose of the zoom center in relation to the (image) pose of at least one reference image point set, wherein the latter includes at least one, but typically more than one, image point. This pose may also be referred to as a reference relative pose. This reference image point set can image in particular at least one point or portion of the calibration object and can have at least one property, in particular which is determinable in an image-based manner. In particular, the property can be a one-to-one property. The property can also be invariant vis-à-vis scaling of the image. In particular, the property allows the reference image point set to be detected in further images of the calibration object, in particular also in the remaining image. The pose of the zoom center in the remaining image can then be determined such that it has the reference relative pose in relation to the reference image point set in the remaining image. The zoom center can be determined as an image coordinate set of a reference point of the reference image point set, wherein the reference image point can be, e.g., a central point of the reference image point set.

This advantageously results in an easily implementable and reliable determination of the pose of the zoom center in the remaining image, which in turn enables an easily implementable and reliable determination of the stereo base, in particular without additional aids still being required for this purpose.

In a further exemplary embodiment, the pose of the zoom center is determined in an object-defined coordinate system. For this purpose, it may be necessary to determine the object-defined coordinate system, in particular its origin and the orientation of axes of this coordinate system, which can be properties of the object-defined coordinate system. An object-defined coordinate system can be a two-dimensional coordinate system. It can be determined in an image-based manner, in particular depending on a property of a point or a portion of the calibration object, said property being determinable in an image-based manner.

For example, at least one property of the object-defined coordinate system can be defined by the reference image point set explained above. Therefore, if the reference image point set is detected in the reference image and also in the remaining image, the object-defined coordinate system can be determined depending on a property of the reference image point set, in particular its pose, wherein the pose of the zoom center is then determined in the object-defined coordinate system determined in this way.

This advantageously results in an easily implementable and reliable determination of the pose of the zoom center in the remaining image, which in turn enables an easily implementable and reliable determination of the stereo base.

In a further exemplary embodiment, the relative pose is changed by changing a pose of the calibration object. In this case, the focal pose cannot be changed or else can be changed simultaneously when the relative pose is changed. A pose of the calibration object can be changed manually. Typically, however, the pose of the calibration object is changed by control of a positioning device for positioning the calibration object for changing the pose of the calibration object. The visualization system can include this positioning device. The positioning device can have, as an end effector, at least one holding device for the calibration object, e.g., a bearing surface, which is movable along at least one linear axis. In this case, the linear axis can be oriented parallel to an optical axis of the stereo system. However, an exact alignment is not mandatory.

This advantageously results in an easily implementable and reliable change of the relative pose, in an automatable manner, which in turn enables an easily implementable and reliable determination of the stereo base.

In a further exemplary embodiment, the relative pose is changed by changing the focal pose. The focal pose can be changed by a movement of at least one optical element of the visualization system. In this exemplary embodiment, the stereo system can be part of a varioscope. Alternatively or cumulatively, the focal pose can be changed by a movement of the microscope head, in particular by drive devices of the stand being controlled in such a way that the pose of the microscope head is changed. This advantageously likewise results in an easily implementable and reliable change of the relative pose, in particular in an automatable manner, which in turn enables an easily implementable and reliable determination of the stereo base. It is possible for a magnification also to be changed at the same time as the focal pose is changed. Typically, however, the magnification remains the same.

In a further exemplary embodiment, the sequence including at least the generating step and the determining step after changing the relative pose is carried out in an automated manner, in particular by an evaluation device. In particular, a trigger signal that causes the sequence to be carried out can be generated after completion of the setting step or attainment of the relative pose to be set. Moreover, a trigger signal that causes a renewed setting step to be carried out can be generated after completion of the determining step. This advantageously results in a temporally rapid and automated determination of the pose of the stereo base.

In one exemplary embodiment, a compensating straight line is determined by the poses of the zoom center in the remaining image, wherein the pose of the stereo base is determined depending on the compensating straight line. In particular, the compensating straight line can be assumed to be a straight line along which the zoom centers progress, as a result of which reference can be made to the above explanations in this regard. This advantageously results in the pose of the stereo base being determined accurately and as independently of noise as possible.

In a further exemplary embodiment, before determining the pose of the zoom center, a distortion correction is carried out in the first image and/or in the further image. This advantageously results in an accurate determination of the pose of the stereo base.

In this case, the proposed method is not necessarily restricted to a stereo system of a medical visualization system and can also be employed for stereo systems used in some other way, e.g., for a stereo system in a vehicle, in an industrial inspection system, in a robot, in a navigation system, in particular a medical navigation system, in a safety or monitoring system, in a portable device (e.g., mobile devices and augmented reality/virtual reality (AR/VR) glasses).

What is further proposed is a medical visualization system including a stereo system having a first and a further image capturing device, at least one means for changing a relative pose between a calibration object and a focal pose of the medical visualization system, and at least one evaluation device. Further, the medical visualization system can include at least one optical system for setting different zoom values for at least one of the image capturing devices. It is possible that the zoom values for both image capturing devices can be set independently of one another. Typically, however, the zoom values are set jointly for both image capturing devices.

The medical visualization system is configured to carry out a method according to any of the exemplary embodiments described in this disclosure.

The means for changing the relative pose can include, e.g., a positioning device for the calibration object and/or an element for changing the focal pose.

The evaluation device can be embodied as at least one computing device or can include at least one such computing device. A computing device in turn can include or be embodied as at least one microcontroller and/or at least one integrated circuit, e.g., a field programmable gate array (FPGA). It is possible that, for this purpose, the evaluation device includes at least one graphics processing unit (GPU). The evaluation device can also be embodied as a control device and control, e.g., the means for changing the relative pose.

The medical visualization system advantageously enables the implementation of a method according to any of the exemplary embodiments described in this disclosure, together with the advantages that have already been explained.

### BRIEF DESCRIPTION OF THE DRAWINGS

The disclosure will now be described with reference to the drawings wherein:

FIG. 1 shows a schematic illustration of a medical visualization system according to an exemplary embodiment of the disclosure,

FIG. 2A shows a schematic illustration of observation beam paths and sensor surfaces in an unaligned pose,

FIG. 2B shows a schematic illustration of observation beam paths and sensor surfaces in an aligned pose,

FIG. 3 shows a schematic illustration of a reference image,

FIG. 4 shows a schematic illustration of a remaining image,

FIG. 5 shows a flowchart of a method according to a first exemplary embodiment of the disclosure,

FIG. 6 shows a schematic illustration of a progression of zoom centers in the remaining image,

FIG. 7 shows a schematic view of an arrangement of observation beam paths and a main objective in a first exemplary embodiment of the disclosure,

FIG. 8 shows a schematic view of an arrangement of observation beam paths and a main objective in a further exemplary embodiment of the disclosure,

FIG. 9 shows a schematic flowchart of a method according to a second exemplary embodiment of the disclosure, and

FIG. 10 shows a schematic illustration of zoom centers in the remaining image.

### DESCRIPTION OF EXEMPLARY EMBODIMENTS

Identical reference signs hereinafter designate elements having identical or similar technical features.

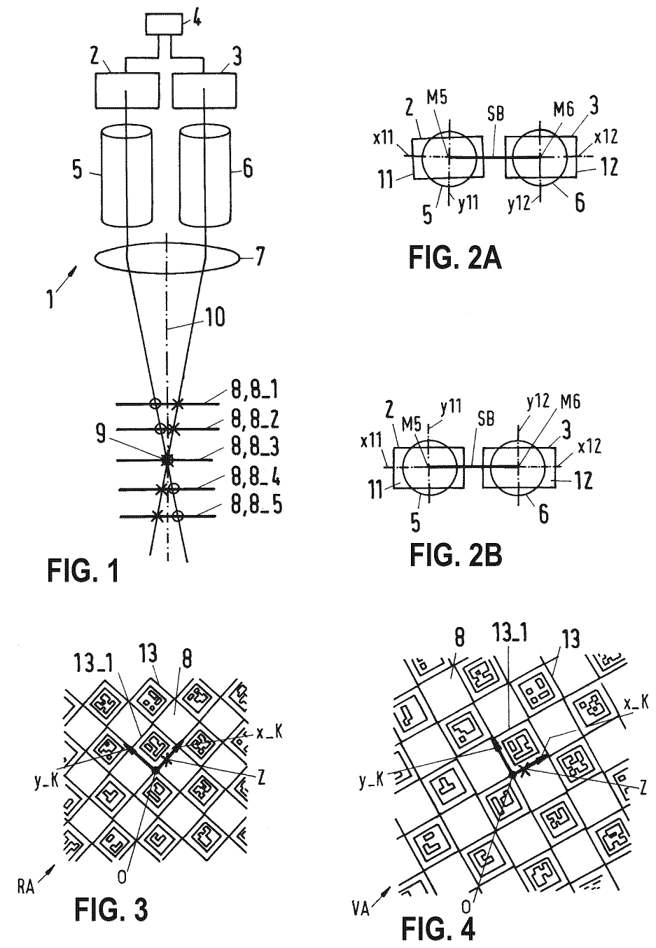

FIG. 1 schematically illustrates a medical visualization system 1 according to an exemplary embodiment of the disclosure, which includes a stereo system having a first image capturing device 2 and a further image capturing device 3, and also an evaluation device 4.

A first observation beam path 5 for the beams captured by the first image capturing device 2 and a further observation beam path 6 for the beams captured by the further image capturing device 3 are schematically illustrated. The illustration likewise shows a main objective 7 and a calibration object 8 in a plurality of spatial poses 8_1, 8_2, 83, 8_4, 8_5, and also a currently set focal point 9 of the visualization system 1. The main objective 7 can have a fixed or an adjustable focal length. Likewise schematically illustrated is an optical axis 10 of the visualization system, which axis can be defined by the main objective 7.

In the different spatial poses 8_1, . . . , 8_5 of the calibration object 8, different relative poses are set between the calibration object 8 and the focal pose of the medical visualization system 1. A cross represents different object points which are imaged into a center, in particular into a central image point set or a central image point, of the image generated by the further image capturing device 3 in the different spatial poses, wherein the central image point set/central image point can form a zoom center of this image. It is evident that different object points are imaged into this center in conjunction with different relative poses. This effect can be used, as explained below, to determine a pose of a stereo base SB of the stereo system (see, e.g., FIG. 2A). In this regard, the represented object points are the relative pose-specific zoom centers of the further image capturing device 3. These can each be back-projected into the image of the first image capturing device 2. A circle represents different object points which are imaged into a center, in particular into a central image point set or a central image point, of the image generated by the first image capturing device 2 in the different spatial poses, wherein the central image point set/central image point can form a zoom center of this image.

FIG. 2A shows a schematic illustration of observation beam paths 5, 6 and sensor surfaces 11, 12 of the image capturing devices 2, 3 of the stereo system in a state in which the sensor surfaces are not aligned according to a target pose. FIG. 2A illustrates a stereo base SB corresponding to a connecting line of centers of the observation beam paths 5, 6 in a cross-sectional plane perpendicular to the central axis M5, M6 of a beam path. In this case, the central axes M5, M6 of the beam paths 5, 6 are oriented parallel at least in portions.

In a target pose of the sensor surfaces 5, 6, which in particular can denote a target orientation, these are arranged in particular such that a vertical axis of symmetry y11, y12 of the respective sensor surface 11, 12 is oriented perpendicularly to the stereo base SB and a horizontal axis of symmetry x11, x12 is oriented parallel to the stereo base SB. In FIG. 2A it can be seen that both the actual pose of the first sensor surface 11 and the actual pose of the further sensor surface 12 deviate from this target pose.

FIG. 2B shows a schematic illustration of observation beam paths 5, 6 and sensor surfaces 11, 12 of the image capturing devices 2, 3 of the stereo system in a state in which the sensor surfaces are aligned according to the target pose explained. In such a state, a simple evaluation and image processing for determining the three-dimensional representation are possible.

FIG. 3 shows a schematic illustration of a reference image RA, which was generated by one of the image capturing devices 2, 3 and images a calibration object 8. The calibration object 8 has a plurality of optically capturable features 13, which are formed as mutually different patterns. Each of these features 13 can be assigned a one-to-one identity. Evaluation of the reference image RA enables these features 13 to be detected and identified. In the exemplary embodiment illustrated in FIG. 3, each feature 13 is formed as a rectangular pattern in the form of a barcode, each pattern having a closed rectangular boundary and differently formed inner portions within the boundary.

The illustration further shows an image point into which a zoom center Z of the image capturing device 2, 3 which generates the reference image RA is imaged. The pose of the zoom center, i.e., image coordinates, can be determined in an object-defined coordinate system having an origin O and coordinate axes x_K, y_K. In this regard, the pose of the zoom center Z can be determined by an x-coordinate and a y-coordinate with respect to the object-defined coordinate system as follows:

$$L\_Z = O + R 1 * x + R 2 * y \qquad formula 1$$

where L_Z denotes the pose of the zoom center Z, R1 denotes the value of the x-coordinate, and R2 denotes the value of the y-coordinate and O denotes the origin.

The origin O and the coordinate axes x_K, y_K oriented at right angles to one another are defined by a reference feature 13_1. In this regard, a bottom left corner of the reference feature 13_1 in the image coordinate system of the reference image RA forms the origin O of the object-defined coordinate system. The orientation of a connecting line from this bottom left corner to a bottom right corner of the reference feature 13_1 defines the orientation of a first coordinate axis x_K and the orientation of a connecting line from the bottom left corner to a top left corner of the reference feature 13_1 defines the orientation of a second coordinate axis y_K. It goes without saying that a different definition of the object-defined coordinate system is also conceivable. Consequently, each feature 13 can define an object-defined coordinate system, which may also be referred to as a feature-specific coordinate system.

The reference feature 13_1 can be determined as that feature of the set of all features 13 which defines an origin O of a feature-specific coordinate system with the smallest distance to the zoom center Z.

FIG. 4 shows a schematic illustration of a remaining image VA, which was generated by the remaining image capturing device 3, 2 and likewise images the calibration object 8. Discernible are the optically capturable features 13 and the reference feature 13_1, which—as explained above—defines the object-defined coordinate system in the remaining image VA.

The image point in the remaining image VA into which the zoom center Z is imaged is likewise illustrated. This image point forms a back-projected zoom center rZ (see FIG. 6). The image coordinates of this image point can be determined such that the pose of the zoom center Z in the object-defined coordinate system which was determined in the remaining image VA is equal to the pose of the zoom center Z in the object-defined coordinate system which was determined in the reference image RA. In particular, the pose can be determined according to formula 1, but with respect to the pose of the object-defined coordinate system in the remaining image VA.

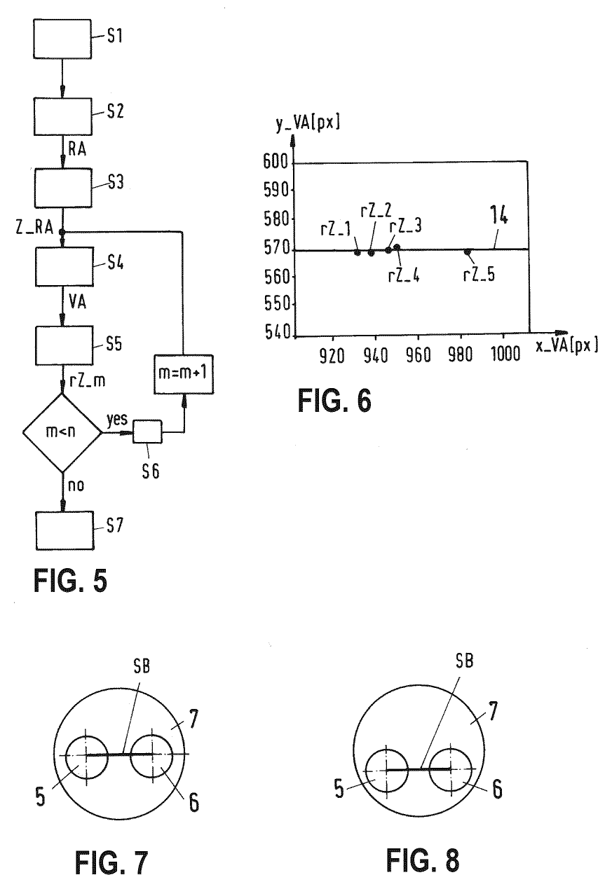

FIG. 5 shows a schematic flowchart of a method according to a first exemplary embodiment of the disclosure. In a setting step S1, a first relative pose between a calibration object 8 (see FIG. 1) and a focal pose of the medical visualization system 1 is set. In this case, the calibration object 8 can be formed as illustrated in FIG. 3.

In a generating step S2, an image is generated as a reference image RA with one of the image capturing devices 2, 3 of the stereo system. The images can be encoded or represented in the form of image signals. One of these images forms a reference image RA and the remaining image forms a remaining image VA. In a first zoom center determining step S3, a reference image-specific zoom center Z_RA in the reference image RA is determined. For this purpose, it may be necessary to generate at least one further image using the image capturing device 2, 3 whose image serves as the reference image RA, wherein the corresponding zoom value is changed for this purpose. The reference image-specific zoom center Z_RA can then be determined by evaluation of these at least two images.

In a further generating step S4, an image is generated as a remaining image VA with the remaining image capturing device 3, 2 of the stereo system. A further determining step S5 then involves determining a pose of a (back)projected zoom center rZ in the remaining image VA. In other words, the pose of the reference image-specific zoom center Z_RA projected into the remaining image VA is determined. This projection of the reference image-specific zoom center Z_RA into the remaining image VA can be determined in an image-based manner, as has already been explained above.

In a change step S6, the relative pose between the calibration object 8 and a focal pose of the medical visualization system 1 is changed at least once, wherein after the or each change or for each set relative pose, the sequence including the further generating step S4 and the further determining step S5 is repeated. In this regard, this sequence can be carried out n times, where n can be larger than one. In the exemplary embodiment illustrated in FIG. 5, n=5. In particular, a counter variable m can be incremented by one after each further determining step S5. After all iterations have been completed, n poses of the (projected) zoom center rZ in the remaining image VA have been determined.

A pose determining step S7 can then involve determining a pose of the stereo base SB in the image coordinate system of the remaining image VA, in particular as an angle between a compensating straight line 14 (see FIG. 6) and a horizontal image axis x_VA.

In this case, the first of the further generating steps S4 can be carried out simultaneously with step S2 for generating the reference image RA. If a plurality of images is required for determining the reference image-specific zoom center Z_RA, these can be generated simultaneously with the further generating steps S4.

FIG. 6 shows a schematic illustration of a progression of (projected) zoom centers rZ in the remaining image VA. An image coordinate system with a horizontal image axis x_VA and a vertical image axis y_VA of the remaining image is illustrated. The illustration shows five zoom centers rZ_1, . . . , rZ_5 arranged at different image point coordinates of the remaining image VA. These image coordinates and hence the pose of the zoom centers rZ_1, . . . , rZ_5 were determined in each further determining step S5 of the method illustrated in FIG. 5. A compensating straight line 14 is furthermore illustrated. This represents a straight line along which the projected zoom centers rZ1, . . . , rZ5 progress.

The pose of this compensating straight line 14, in particular its gradient, represents information about the pose of the stereo base SB (see FIG. 2A) in the corresponding image coordinate system of the remaining image VA. If the gradient is zero and the compensating straight line thus runs parallel to the horizontal image axis x_VA, the pose of the stereo base SB can correspond to a target pose. In this target pose of the stereo base SB—as explained above—the sensor surfaces 12, 13 of the image capturing devices 2, 3 also have a target pose, in particular the sensor surface 12, 13 of the image capturing device 2, 3 which generates the remaining image VA. If the gradient is different than zero and the compensating straight line 14 therefore does not run parallel to the horizontal image axis x_VA, the corresponding sensor surfaces 12, 13 cannot be arranged in their respective target pose, in particular the sensor surface 12, 13 of the image capturing device 2, 3 which generates the remaining image VA. A user can then change a pose, in particular an orientation, of the sensor surfaces 12, 13 until the gradient of the compensating straight line 14 has the value zero. In particular, for this purpose, at least one of the sensor surfaces 12, 13, namely the sensor surface 12, 13 of the image capturing device 2, 3 which generates the remaining image VA, can be rotated about a rotation axis which is oriented orthogonally to the sensor surface 12, 13 and runs through the zoom center Z, in particular by the gradient angle of the compensating straight line 14. Alternatively, the angle/gradient can be taken into account when determining the three-dimensional representation, i.e., in a reconstruction method.

FIG. 7 shows a schematic view of an arrangement of observation beam paths 5, 6 and a main objective 7 in a first exemplary embodiment. The illustration shows that a stereo base SB is arranged on one of the central axes of symmetry of the main objective 7 or on one of the central axes of symmetry of a beam path for the beams passing through the main objective 7. This advantageously results in as complete utilization as possible of the capture range defined by the main objective 7.

FIG. 8 shows a schematic view of an arrangement of observation beam paths 5, 6 and a main objective 7 in a further exemplary embodiment. The illustration shows that a stereo base SB is arranged spaced apart from one of the central axes of symmetry of the main objective 7 or spaced apart from one of the central axes of symmetry of a beam path for the beams passing through the main objective 7. In this case, however, the stereo base SB is oriented parallel to such an axis of symmetry. This advantageously results in space for the arrangement of an illumination device, in particular a field of view illumination device, which illuminates an examination region through the main objective 7.

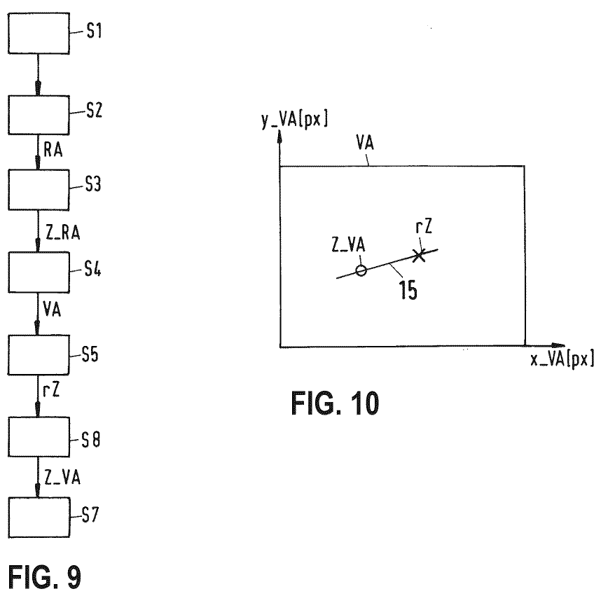

FIG. 9 shows a schematic flowchart of a method according to a second exemplary embodiment of the disclosure. As in the exemplary embodiment illustrated in FIG. 5, a sequence including a setting step S1, a generating step S2, a first zoom center determining step S3 and a further generating step S4 is carried out.

In contrast to the exemplary embodiment illustrated in FIG. 5, a further zoom center determining step S8 is additionally carried out as well. This step involves determining an image-specific zoom center Z_VA in the remaining image VA. This is illustrated in FIG. 10. A further determining step S5 involves determining a (back)projected zoom center rZ as a projection of the reference image-specific zoom center Z_RA determined in the first zoom center determining step S3 into the remaining image VA. This, too, is illustrated in FIG. 10. FIG. 9 illustrates that the further determining step S5 is carried out before the further zoom center determining step S8. However, this is not mandatory. The steps can also be carried out in a different order. In a pose determining step S7, it is possible to determine a pose of the stereo base SB in the image coordinate system of the remaining image VA, in particular as an angle between a connecting straight line 15 (see FIG. 10) and a horizontal image axis x_VA, wherein the connecting straight line 15 connects the image-specific zoom center Z_VA and the (back)projected zoom center rZ.

FIG. 10 shows a schematic illustration of the image-specific zoom center Z_VA determined for the remaining image VA and the reference image-specific zoom center rZ projected into the remaining image. The illustration likewise shows a horizontal image axis x_VA and a connecting straight line 15, which connects both zoom centers Z_VA, rZ in the remaining image VA. This connecting straight line 15 represents a pose of the stereo base SB (see FIG. 2B) in the image coordinate system of the remaining image VA.

### LIST OF REFERENCE NUMERALS

1 visualization system
2 first image capturing device
3 further image capturing device
4 evaluation device
first observation beam path
6 further observation beam path
7 main objective
8 calibration object
8_1, . . . 8_5 spatial poses
9 focal point
optical axis
11 first sensor surface
12 further sensor surface
13 features
13_1 reference feature
14 compensating straight line
connecting straight line
O origin
RA reference image
SB stereo base
VA remaining image
Z zoom center
Z_RA reference image-specific zoom center
Z_VA image-specific zoom center
rZ (back)projected zoom center
rZ_1, . . . rZ_5 (back)projected zoom centers
S1 setting step
S2 generating step
S3 first zoom center determining step
S4 further generating step
S5 further determining step
S6 change step
S7 pose determining step
S8 further zoom center determining step
y_K, x_K coordinate axes
x11, y11 axis of symmetry
x12, y12 axis of symmetry

1. A method for determining a pose of a stereo base of a stereo system of a medical visualization system, the medical visualization system including a first image capturing device and a second image capturing device, the stereo base being a connecting line between centers of beam paths of the first and second image capturing devices in cross-sectional planes perpendicular to center axes of the beam paths, the method comprising:
(a) setting a first relative pose between a calibration object and a focal pose of the medical visualization system;
(b) generating a first image with one of the first and second image capturing devices of the stereo system, wherein the first image serves as a reference image;
(c) determining a reference image-specific zoom center in the reference image;
(d) generating a second image with a further image capturing device of the first and second image capturing devices of the stereo system, wherein the second image serves as a remaining image;
(e) determining a pose of a back-projected zoom center as a pose of a reference image-specific zoom center back-projected into the remaining image, wherein a pixel or a set of pixels is/are determined in the remaining image which represent(s) a point or a section of the calibration object, respectively, that is mapped into the reference image-specific zoom center of the reference image,
wherein:
(f) the relative pose is changed at least once and steps (d) to (e) are carried out for each relative pose that is changed, wherein the pose of the stereo base is determined depending on the poses of the back-projected zoom center in the remaining image, or
(g) the pose of an image-specific zoom center in the remaining image is determined, wherein the pose of the stereo base is determined depending on the poses of the image-specific zoom center and the back-projected zoom center in the remaining image.

2. The method as claimed in claim 1, wherein at least two images are generated by the one of the first and second image capturing devices which generates the reference image,
wherein the at least two images are generated with different zoom values in each case, and
wherein the reference image-specific zoom center is determined by evaluating the at least two images.

3. The method as claimed in claim 1, wherein a pose of the reference image-specific zoom center in the reference image is determined, and
wherein determining the pose of the back-projected zoom center is carried out depending on the pose in the reference image.

4. The method as claimed in claim 1, wherein the pose of the zoom center is determined in an object-defined coordinate system.

5. The method as claimed in claim 4, wherein a pose of the object-defined coordinate system is determined in an image-based manner.

6. The method as claimed in claim 1, wherein the relative pose is changed by changing a pose of the calibration object.

7. The method as claimed in claim 1, wherein the relative pose is changed by changing the focal pose.

8. The method as claimed in claim 1, wherein carrying out at least steps (d) to (e) is carried out in an automated manner after changing the relative pose.

9. The method as claimed in claim 1, wherein a compensating straight line is determined by the poses of the zoom center in the remaining image, and
wherein the pose of the stereo base is determined depending on the compensating straight line.

10. The method as claimed in claim 1, wherein before determining the pose of the zoom center, a distortion correction is carried out in the first image and/or in the further image.

11. A medical visualization system, comprising:
the stereo system including the first image capturing device and the second image capturing device;
at least one means for changing a relative pose between a calibration object and a focal pose of the medical visualization system; and
at least one evaluation device, wherein the medical visualization system is configured to carry out the method as claimed in claim 1.
